(19) United States
(12) Patent Application Publication
(10) Pub. No.: US 2026/0294519 A1
(43) Pub. Date: Oct. 1, 2026

(54) SURGICAL END EFFECTOR ASSEMBLIES AND SURGICAL INSTRUMENTS FOR ENERGY-BASED TISSUE CUTTING

(71) Applicant: Covidien LP, Mansfield, MA (US)

(72) Inventors: Craig V. Krastins, Arvada, CO (US); Chelsea E. Walbridge, Longmont, CO (US); Daniel Wayne Mercier, Erie, CO (US); Zoe M. Schmanski, Broomfield, CO (US); Jake H. Young, Superior, CO (US); James D. Allen, Broomfield, CO (US); Dylan Richard Kingsley, Broomfield, CO (US); David J. Van Tol, Boulder, CO (US)

(21) Appl. No.: 19/489,808

(22) PCT Filed: Jun. 3, 2024
(86) PCT No.: PCT/IB2024/055414

§ 371 Date: Dec. 3, 2025

Related U.S. Application Data
(60) Provisional application No. 63/471,469, filed on Jun. 6, 2023.

(51) Int. Cl. A61B 18/14; A61B 18/00; A61B 34/30

(52) U.S. Cl. CPC A61B 18/1445; A61B 34/30; A61B 2018/00601; A61B 2018/0063; A61B 2018/1452

(57) ABSTRACT

A surgical end effector assembly includes first and second jaw members each having a tissue contacting surface. At least one of the jaw members is movable relative to the other between a spaced apart position and an approximated position. The second jaw member includes a cutting electrode extending from the second jaw member towards the first jaw member. The first jaw member includes a jaw body defining a slot. At least one retainer, at least one overhang of a tissue contacting plate, and/or at least one channel in communication with the slot facilitates engagement and retention of a compression pad at least partially within the slot. In the approximated position, the cutting electrode and the compression pad are configured to grasp tissue therebetween.

### CROSS-REFERENCE TO RELATED APPLICATION

This application claims the benefit of U.S. Provisional Patent Application Ser. No. 63/471,469, filed Jun. 6, 2023, the entire content of which is incorporated herein by reference.

### FIELD

This disclosure relates to surgical instruments and, more specifically, to surgical end effector assemblies and surgical instruments for energy-based tissue cutting such as, for example, for use in surgical robotic systems.

### BACKGROUND

Surgical robotic systems are increasingly utilized in various different surgical procedures. Some surgical robotic systems include a console supporting a robotic arm. One or more different surgical instruments may be configured for use with the surgical robotic system and selectively mountable to the robotic arm. The robotic arm provides one or more inputs to the mounted surgical instrument to enable operation of the mounted surgical instrument.

A surgical forceps, one type of instrument capable of being utilized with a robotic surgical system, relies on mechanical action between its jaw members to grasp, clamp, and constrict tissue. Electrosurgical forceps utilize both controlled mechanical clamping action and energy to heat tissue to seal (or otherwise treat) tissue. Typically, once tissue is sealed, the tissue is severed using a cutting element. Accordingly, many electrosurgical forceps are designed to incorporate a mechanical cutting element to effectively sever sealed tissue (and/or to cut tissue independently of tissue sealing). Alternatively, surgical forceps may incorporate an energy-based, e.g., thermal, electrical, ultrasonic, etc., cutting mechanism to cut tissue, whether previously sealed or unsealed.

### SUMMARY

As used herein, the term “distal” refers to the portion that is being described which is farther from an operator (whether a human surgeon or a surgical robot), while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, design variations, and/or other variations, up to and including plus or minus 10 percent. To the extent consistent, any of the aspects described herein may be used in conjunction with any or all of the other aspects described herein.

Provided in accordance with aspects of this disclosure is a surgical end effector assembly having first and second jaw members including respective first and second tissue contacting surfaces. At least one of the first or second jaw members is movable relative to the other of the first or second jaw members between a spaced apart position and an approximated position. The second jaw member includes a cutting electrode extending from the second jaw member towards the first jaw member. The first jaw member includes a jaw body defining a slot and at least one retainer fixed relative to the jaw body and extending into the slot. The first jaw member further includes a compression pad at least partially disposed within the slot and about the at least one retainer to capture at least a portion of the at least one retainer within the compression pad and engage the compression pad at least partially within the jaw body. In the approximated position, the cutting electrode and the compression pad are configured to grasp tissue therebetween.

In an aspect of this disclosure, the at least one retainer includes a first retainer extending inwardly into the slot from a first side of the slot and a second retainer extending inwardly into the slot from a second, opposite side of the slot. In such aspects, the at least one retainer may include a plurality of first retainers and/or a plurality of second retainers.

In another aspect of this disclosure, each of the first and second retainers includes a body extending substantially longitudinally through the slot. The bodies of the first and second retainers are captured within the compression pad.

In yet another aspect of this disclosure, the at least one retainer includes a body extending substantially transversely across the slot. The body of the at least one retainer is captured within the compression pad.

In still another aspect of this disclosure, the at least one retainer varies an effective durometer of the compression pad in at least one dimension thereof.

In still yet another aspect of this disclosure, the jaw body of the first jaw member includes a jaw housing and a jaw insert disposed within the jaw housing. The jaw insert defines the slot and the at least one retainer is fixed relative to the jaw insert.

In another aspect of this disclosure, the at least one retainer includes at least one wire.

Another surgical end effector assembly provided in accordance with this disclosure has first and second jaw members including respective first and second tissue contacting surfaces. At least one of the first or second jaw members is movable relative to the other of the first or second jaw members between a spaced apart position and an approximated position. The second jaw member includes a cutting electrode extending from the second jaw member towards the first jaw member. The first jaw member includes a jaw body defining a slot, a tissue contacting plate defining the first tissue contacting surface and extending along first and second longitudinal sides of the slot, and a compression pad. First and second overhangs extend from the tissue contacting plate into the slot from the respective first and second longitudinal sides of the slot. The compression pad is at least partially disposed within the slot. In the approximated position, the cutting electrode and the compression pad are configured to grasp tissue therebetween.

In an aspect of this disclosure, the compression pad captures the first and second overhangs therein and extends over a portion of the first tissue contacting surface.

In another aspect of this disclosure, the first and second overhangs extend uninterrupted from the tissue contacting plate in coplanar relation relative to the first tissue contacting surface.

In still another aspect of this disclosure, the first and second overhangs are tangs angled relative to the first tissue contacting surface. Alternatively or additionally, the tangs define a reduced thickness compared to the tissue contacting plate.

In yet another aspect of this disclosure, the first and second overhangs vary an effective durometer of the compression pad in at least one dimension thereof.

Another surgical end effector assembly provided in accordance with this disclosure has first and second jaw members including respective first and second tissue contacting surfaces. At least one of the first or second jaw members is movable relative to the other of the first or second jaw members between a spaced apart position and an approximated position. The second jaw member includes a cutting electrode extending from the second jaw member towards the first jaw member. The first jaw member includes a jaw body defining a slot and at least one channel in communication with the slot. The first jaw member further includes a compression pad at least partially disposed within the slot and the at least one channel to engage the compression pad at least partially within the jaw body. In the approximated position, the cutting electrode and the compression pad are configured to grasp tissue therebetween.

In an aspect of this disclosure, the at least one channel includes at least one channel extending from a bottom of the slot. In aspects, the at least one channel includes a plurality of channels extending from a bottom of the slot in longitudinally spaced relation relative to one another.

In another aspect of this disclosure, the at least one channel includes at least one channel extending from a side of the slot. In aspects, the at least one channel includes a pair of channels extending from opposing sides of the slot.

In still another aspect of this disclosure, the at least one channel varies an effective durometer of the compression pad in at least one dimension thereof.

### BRIEF DESCRIPTION OF THE DRAWINGS

The above and other aspects and features of this disclosure will become more apparent in view of the following detailed description when taken in conjunction with the accompanying drawings wherein like reference numerals identify similar or identical elements.

FIG. 1 is a schematic illustration of a surgical robotic system including a control tower, a console, and one or more surgical robotic arms according to aspects of this disclosure;

FIG. 2 is a perspective view of a surgical robotic arm of the surgical robotic system of FIG. 1 according to aspects of this disclosure;

FIG. 3 is a perspective view of a setup arm with the surgical robotic arm of the surgical robotic system of FIG. 1 according to aspects of this disclosure;

FIG. 4 is a schematic diagram of a computer architecture of the surgical robotic system of FIG. 1 according to aspects of this disclosure;

FIG. 5 is a perspective view of a surgical instrument provided in accordance with the present disclosure configured for mounting on a robotic arm of a surgical robotic system such as the surgical robotic system of FIG. 1;

FIGS. 6A and 6B are front and rear perspective views, respectively, of a proximal portion of the surgical instrument of FIG. 5, with an outer shell removed;

FIG. 7 is a front perspective view of the proximal portion of the surgical instrument of FIG. 5 with the outer shell and additional internal components removed;

FIGS. 8A and 8B are side views of a portion of the end effector assembly of the surgical instrument of FIG. 5 with jaw members of the end effector assembly disposed in spaced apart and approximated positions, respectively;

FIG. 9 is a transverse cross-sectional view of the end effector assembly of the surgical instrument of FIG. 5 with the jaw members of the end effector assembly disposed in the approximated position;

FIGS. 10A-10C are transverse cross-sectional, side longitudinal cross-sectional, and top longitudinal cross-sectional views of a distal portion of one of the jaw members of the end effector assembly of the surgical instrument of FIG. 5 illustrating engagement of a compression pad within the jaw member in accordance with aspects of this disclosure;

FIGS. 11-14 are top longitudinal cross-sectional views of a distal portion of one of the jaw members of the end effector assembly of the surgical instrument of FIG. 5 illustrating engagement of a compression pad within the jaw member in accordance with various other aspects of this disclosure;

FIGS. 15A and 15B are transverse cross-sectional and side longitudinal cross-sectional views, respectively, of a distal portion of one of the jaw members of the end effector assembly of the surgical instrument of FIG. 5 illustrating engagement of a compression pad within the jaw member in accordance with still yet other aspects of this disclosure; and

FIGS. 16-18 are transverse cross-sectional views of a distal portion of one of the jaw members of the end effector assembly of the surgical instrument of FIG. 5 illustrating engagement of a compression pad within the jaw member in accordance with various other aspects of this disclosure.

### DETAILED DESCRIPTION

This disclosure provides surgical end effector assemblies and surgical instruments for energy-based tissue cutting. As described in detail below, the surgical end effector assemblies and surgical instruments of this disclosure are configured for use with a surgical robotic system, which may include, for example, a surgical console, a control tower, and one or more movable carts having a surgical robotic arm coupled to a setup arm. The surgical console receives user input through one or more interface devices, which are interpreted by the control tower as movement commands for moving the surgical robotic arm. The surgical robotic arm includes a controller, which is configured to process the movement command and to generate a torque command for activating one or more actuators of the robotic arm, which, in turn, move the robotic arm in response to the movement command. Those skilled in the art will understand that this disclosure, although described in connection with surgical robotic systems, may also be adapted for use with handheld surgical instruments such as, for example, endoscopic surgical instruments and/or open surgical instruments, whether manually operated or powered.

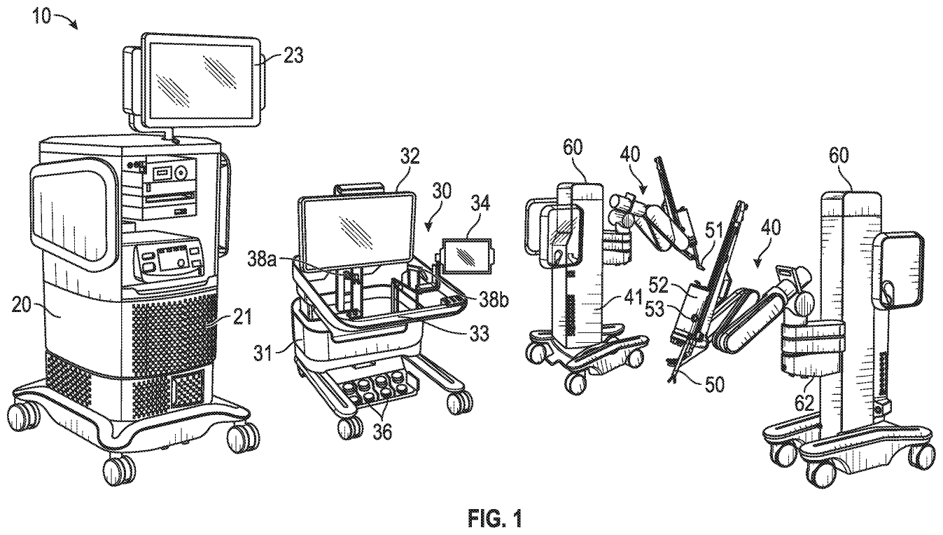

With reference to FIG. 1, a surgical robotic system 10 includes a control tower 20, which is connected to components of the surgical robotic system 10 including a surgical console 30 and one or more robotic arms 40. Each of the robotic arms 40 includes a surgical instrument 50, 51 removably coupled thereto. Each of the robotic arms 40 is also coupled to a movable cart 60.

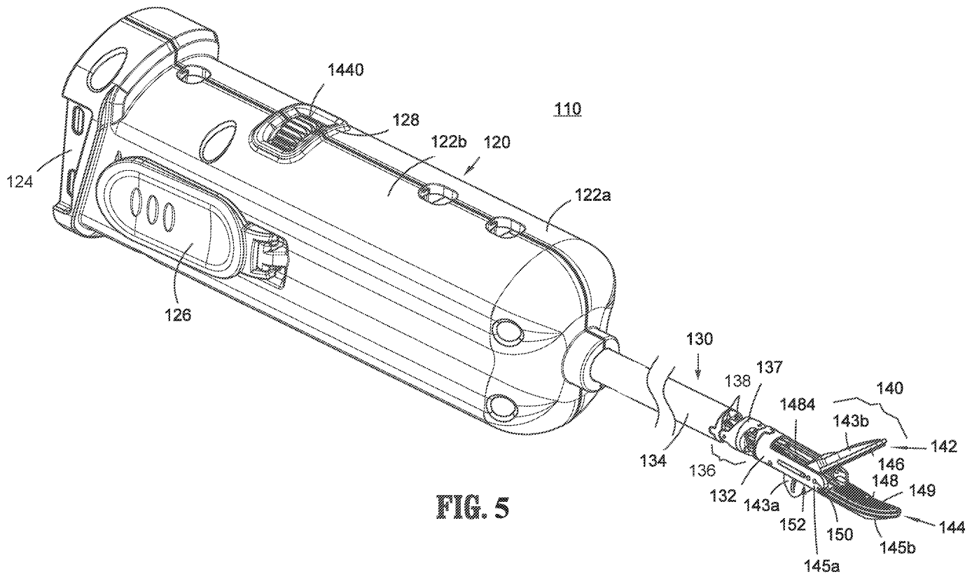

The one or more surgical instruments 50, 51 may be configured for use during minimally invasive surgical procedures and/or open surgical procedures. In aspects, one of the surgical instruments 50 may be an endoscope, such as an endoscope camera 51, configured to provide a video feed for the clinician. In aspects, one of the surgical instruments 50 may be an energy based surgical instrument such as, for example, an energy-based forceps configured to seal tissue by grasping tissue between opposing structures and applying energy, e.g., electrical, thermal, ultrasonic, light, etc., energy thereto and to cut tissue by applying energy, e.g., electrical, thermal, ultrasonic, light, etc., energy thereto. An example of such an energy-based forceps for energy-based sealing and cutting is described in detail below and identified by reference numeral 110 (FIG. 5).

Endoscope camera 51 is configured to capture video of the surgical site. The surgical console 30 includes a first display 32, which displays a video feed of the surgical site provided by endoscope camera 51, and a second display 34, which displays a user interface for controlling the surgical robotic system 10. The first and second displays 32 and 34 are touchscreens allowing for display of and interaction with various graphical user inputs.

The surgical console 30 also includes a plurality of user interface devices, such as foot pedals 36 and a pair of handle controllers 38a and 38b which are used by a user to remotely control robotic arms 40. The surgical console further includes an armrest 33 used to support clinician's arms while operating the handle controllers 38a and 38b.

The control tower 20 includes a display 23, which may be a touchscreen, and outputs on the graphical user interfaces (GUIs). The control tower 20 also acts as an interface between the surgical console 30 and one or more robotic arms 40. In particular, the control tower 20 is configured to control the robotic arms 40, such as to move the robotic arms 40 and the corresponding surgical instrument 50, 51 based on a set of programmable instructions and/or input commands from the surgical console 30, in such a way that robotic arms 40 and the surgical instruments 50, 51 execute a desired movement sequence in response to input from the foot pedals 36 and the handle controllers 38a and 38b.

Each of the control tower 20, the surgical console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area network, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth® (an open wireless protocol for exchanging data over short distances, using short length radio waves, from fixed and mobile devices, creating personal area networks (PANs)), ZigBee® (a specification for a suite of high level communication protocols using small, low-power digital radios based on the IEEE 122.15.4-2003 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

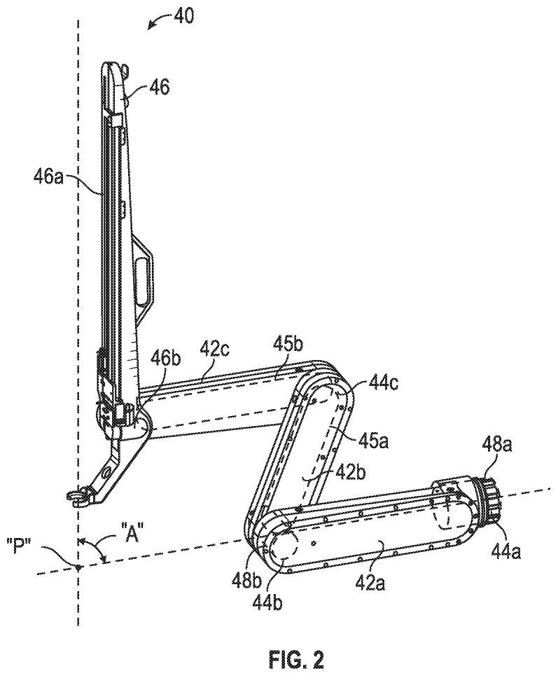

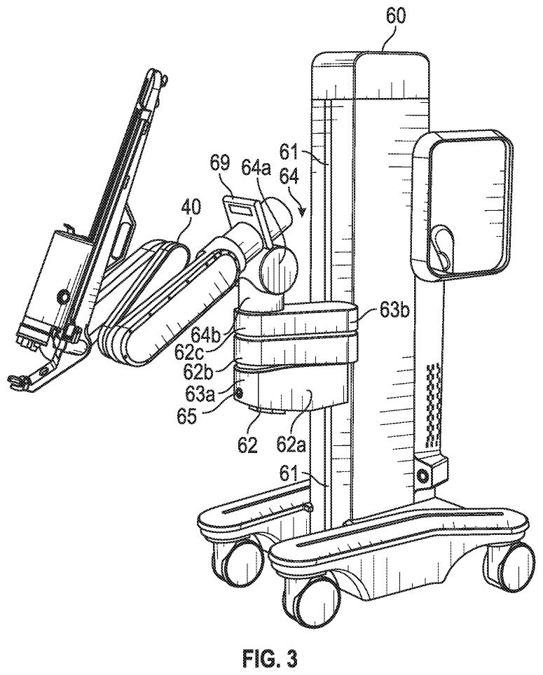

With reference to FIGS. 2 and 3, each of the robotic arms 40 may include a plurality of links 42a, 42b, 42c, which are interconnected at joints 44a, 44b, 44c, respectively. Joint 44a is configured to secure the robotic arm 40 to the movable cart 60 and defines a first longitudinal axis. The movable cart 60 includes a lift 61 and a setup arm 62, which provides a base for mounting of the robotic arm 40. The lift 61 allows for vertical movement of the setup arm 62. The movable cart 60 also includes a display 69 for displaying information pertaining to the robotic arm 40.

The setup arm 62 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62a and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In aspects, the robotic arm 40 may be coupled to the surgical table (not shown). The setup arm 62 includes controls 65 for adjusting movement of the links 62a, 62b, 62c as well as the lift 61.

The third link 62c includes a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

With reference again to FIGS. 1 and 2, the robotic arm 40 also includes a holder 46 defining a second longitudinal axis and configured to receive an IDU 52. The IDU 52 is configured to couple to an actuation mechanism of the surgical instrument 50 and the endoscope camera 51 and is configured to move (e.g., rotate) and actuate the instrument 50 and/or the endoscope camera 51. IDU 52 transfers actuation forces from its actuators to the surgical instrument 50 and/or the endoscope camera 51 to actuate components (e.g., end effectors) of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c.

The robotic arm 40 further includes a plurality of manual override buttons 53 disposed on the IDU 52 and/or the setup arm 62, which may be used in a manual mode. The clinician may press one or the buttons 53 to move the component associated with the button 53.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

The actuator 48b of the joint 44b is coupled to the joint 44c via the belt 45a, and the joint 44c is in turn coupled to the joint 46c via the belt 45b. Joint 44c may include a transfer case coupling the belts 45a and 45b, such that the actuator 48b is configured to rotate each of the links 42b, 42c and the holder 46 relative to each other. More specifically, links 42b, 42c, and the holder 46 are passively coupled to the actuator 48b which enforces rotation about a remote center point “P” which lies at an intersection of the first axis defined by the link 42a and the second axis defined by the holder 46. Thus, the actuator 48b controls the angle “A” between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle “A.” In aspects, some or all of the joints 44a, 44b, 44c may include an actuator to obviate the need for mechanical linkages.

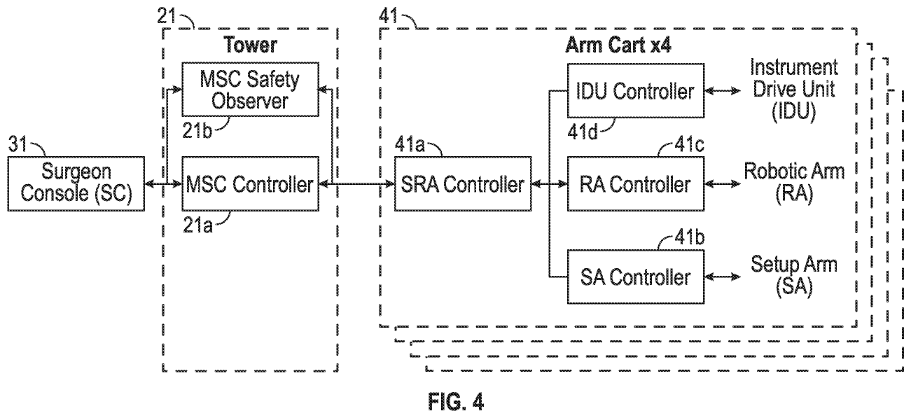

With reference to FIGS. 1 and 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgical console 30 about the current position and/or orientation of the handle controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives back the actual joint angles and uses this information to determine force feedback commands that are transmitted back to the computer 31 of the surgical console 30 to provide haptic feedback through the handle controllers 38a and 38b. The handle controllers 38a and 38b include one or more haptic feedback vibratory devices that output haptic feedback. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 4 la, a setup arm controller 41b, a robotic arm controller 41c, and an IDU controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the movable cart 60, the robotic arm 40, and the IDU 52. The main cart controller 41a also communicates actual joint angles back to the controller 21a.

With additional reference to FIGS. 2 and 3, the setup arm controller 41b controls each of joints 63a and 63b, and the rotatable base 64 of the setup arm 62 and calculates desired motor movement commands (e.g., motor torque) for the pitch axis and controls the brakes. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled as follows. Initially, a pose of the handle controller controlling the robotic arm 40, e.g., the handle controller 38a, is transformed into a desired pose of the robotic arm 40 through a hand eye transform function executed by the controller 21a. The hand eye function, as well as other functions described herein, is/are embodied in software executable by the controller 21a or any other suitable controller described herein. The pose of the handle controller 38a may be embodied as a coordinate position and role-pitch-yaw (“RPY”) orientation relative to a coordinate reference frame, which is fixed to the surgical console 30. The desired pose of the instrument 50 is relative to a fixed frame on the robotic arm 40. The pose of the handle controller 38a is then scaled by a scaling function executed by the controller 21a. In aspects, the coordinate position is scaled down and the orientation is scaled up by the scaling function. In addition, controller 21a also executes a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

Turning to FIGS. 5-7, a surgical instrument 110 provided in accordance with the present disclosure generally includes a housing 120, a shaft 130 extending distally from housing 120, an end effector assembly 140 extending distally from shaft 130, and an actuation assembly 1100 disposed within housing 120 and operably associated with end effector assembly 140. Instrument 110 is detailed herein as an articulating electrosurgical forceps configured for use with a surgical robotic system, e.g., surgical robotic system 10 (FIG. 1). However, the aspects and features of instrument 110 provided in accordance with the present disclosure, detailed below, are equally applicable for use with other suitable surgical instruments, e.g., graspers, staplers, clip appliers, and/or in other suitable surgical systems, e.g., motorized, other power driven systems, and/or manually actuated surgical systems (including handheld instruments).

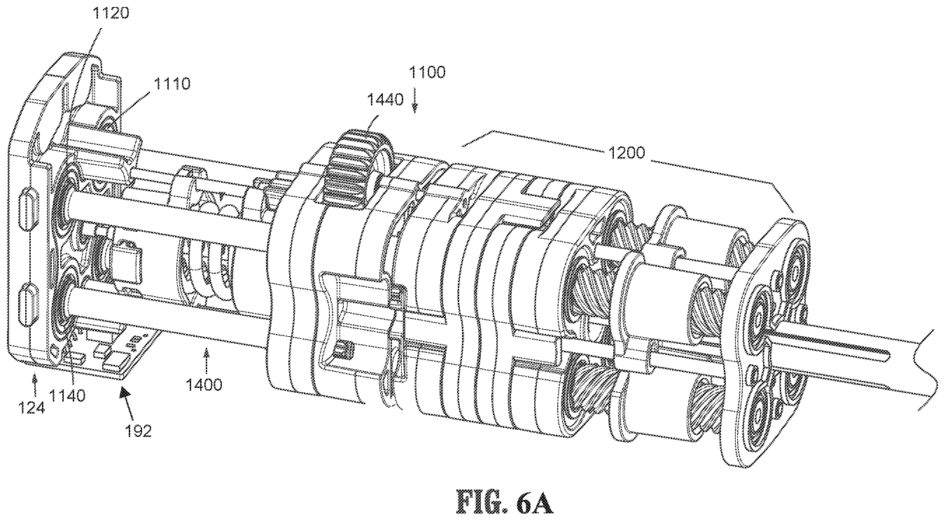

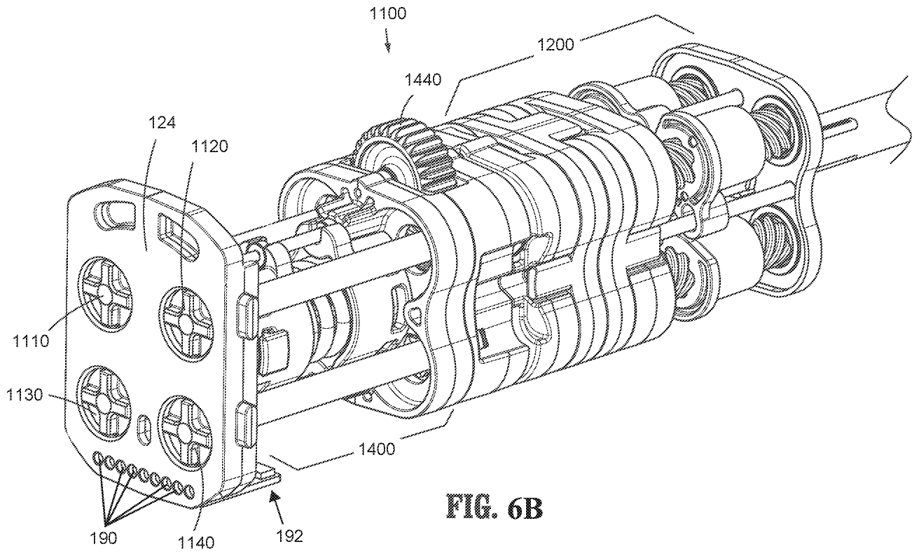

With particular reference to FIG. 5, housing 120 of instrument 110 includes first and second housing parts 122a, 122b and a proximal face plate 124 that cooperate to enclose actuation assembly 1100 therein. Proximal face plate 124 includes through holes defined therein through which input couplers 1110-1140 (FIG. 6B) of actuation assembly 1100 extend. A pair of latch levers 126 (only one of which is illustrated in FIG. 5) extending outwardly from opposing sides of housing 120 enable releasable engagement of housing 120 with a robotic arm 40 (FIG. 1) of a surgical robotic system, e.g., surgical robotic system 10 (FIG. 1). A window 128 defined through housing 120 permits thumbwheel 1440 to extend therethrough to enable manual manipulation of thumbwheel 1440 from the exterior of housing 120 to permit manual opening and closing of end effector assembly 140.

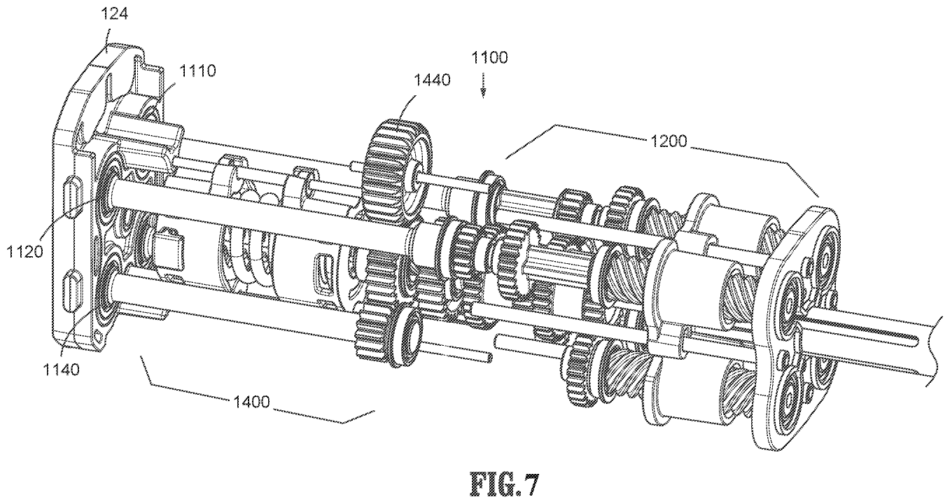

Referring also to FIGS. 6A-7, a plurality of electrical contacts 190 extend through one or more apertures defined through proximal face plate 124 to enable electrical communication between instrument 110 and surgical robotic system 10 (FIG. 1) when instrument 110 is engaged on a robotic arm thereof, e.g., for the communication of data, control, and/or power signals therebetween. As an alternative to electrical contacts 190 extending through proximal face plate 124, other suitable transmitter, receiver, and/or transceiver components to enable the communication of data, control, and/or power signals are also contemplated, e.g., using RFID, Bluetooth®, WiFi®, or via any other suitable wired, wireless, contacted, or contactless communication method. At least some of the electrical contacts 190 are electrically coupled with electronics 192 mounted on an interior side of proximal face plate 124, e.g., within housing 120. Electronics 192 may include, for example, a storage device, a communications device (including suitable input/output components), and a CPU including a memory and a processor. Electronics 192 may be mounted on a circuit board or otherwise configured, e.g., as a chip.

The storage device of electronics 192 stores information relating to surgical instrument such as, for example: the item number, e.g., SKU number; date of manufacture; manufacture location, e.g., location code; serial number; lot number; use information; setting information; adjustment information; calibration information; security information, e.g., encryption key(s), and/or other suitable additional or alternative data. The storage device of electronics 192 may be, for example, a magnetic disk, flash memory, optical disk, or other suitable data storage device.

As an alternative or in addition to storing the above noted information in the storage device of electronics 192, some or all of such information, e.g., the use information, calibration information, setting information, and/or adjustment information, may be stored in a storage device associated with surgical robotic system 10 (FIG. 1), a remote server, a cloud server, etc., and accessible via instrument 110 and/or surgical robotic system 10 (FIG. 1). In such configurations, the information may, for example, be updated by manufacturer provided updates, and/or may be applied to individual instruments, units of instruments (e.g., units from the same manufacturing location, manufacturing period, lot number, etc.), or across all instruments. Further still, even where the information is stored locally on each instrument, this information may be updated by manufacturer provided updates manually or automatically upon connection to the surgical robotic system 10 (FIG. 1).

Referring again to FIG. 5, shaft 130 of instrument 110 includes a distal clevis segment 132, a proximal segment 134, and an articulating section 136 disposed between the distal clevis and proximal segments 132, 134, respectively. Articulating section 136 includes one or more articulating components 137, e.g., links, joints, etc. A plurality of articulation cables 138, e.g., four (4) articulation cables, or other suitable actuators, extend through articulating section 136. More specifically, articulation cables 138 are operably coupled to distal clevis segment 132 of shaft 130 at the distal ends thereof and extend proximally from distal clevis segment 132 of shaft 130, through articulating section 136 of shaft 130 and proximal segment 134 of shaft 130, and into housing 120, wherein articulation cables 138 operably couple with an articulation sub-assembly 1200 of actuation assembly 1100 (FIG. 6A) to enable selective articulation of distal clevis segment 132 (and, thus end effector assembly 140) relative to proximal segment 134 and housing 120, e.g., about at least two axes of articulation (yaw and pitch articulation, for example). Articulation cables 138 are arranged in a generally rectangular configuration, although other suitable configurations are also contemplated. In some configurations, as an alternative, shaft 130 is substantially rigid, malleable, or flexible and not configured for active articulation. Articulation sub-assembly 1200 is described in greater detail below.

With respect to articulation of end effector assembly 140 relative to proximal segment 134 of shaft 130, actuation of articulation cables 138 may be accomplished in pairs. More specifically, in order to pitch end effector assembly 140, the upper pair of cables 138 are actuated in a similar manner while the lower pair of cables 138 are actuated in a similar manner relative to one another but an opposite manner relative to the upper pair of cables 138. With respect to yaw articulation, the right pair of cables 138 are actuated in a similar manner while the left pair of cables 138 are actuated in a similar manner relative to one another but an opposite manner relative to the right pair of cables 138. Other configurations of articulation cables 138 or other articulation actuators are also contemplated.

Continuing with reference to FIG. 5, end effector assembly 140 includes first and second jaw members 142, 144, respectively. Each jaw member 142, 144 includes a proximal flange 143a, 145a and a distal body 143b, 145b, respectively. Distal bodies 143b, 145b define opposed tissue contacting surfaces 146, 148, respectively. Proximal flanges 143a, 145a are pivotably coupled to one another about a pivot 150 and are operably coupled to one another via a cam slot assembly 152 including a cam pin slidably received within cam slots defined within the proximal flange 143a, 145a of at least one of the jaw members 142, 144, respectively, to enable pivoting of jaw member 142 relative to jaw member 144 and distal segment 132 of shaft 130 between a spaced apart position (e.g., an open position of end effector assembly 140) and an approximated position (e.g., a closed position of end effector assembly 140) for grasping tissue between tissue contacting surfaces 146, 148. As an alternative to this unilateral configuration, a bilateral configuration may be provided whereby both jaw members 142, 144 are pivotable relative to one another and distal segment 132 of shaft 130. Alternatively, the above detailed configuration may be reversed, e.g., wherein jaw member 142 is the fixed jaw member and jaw member 144 is movable relative to jaw member 142. Other suitable jaw actuation mechanisms (for bilateral and/or unilateral jaw configurations) are also contemplated.

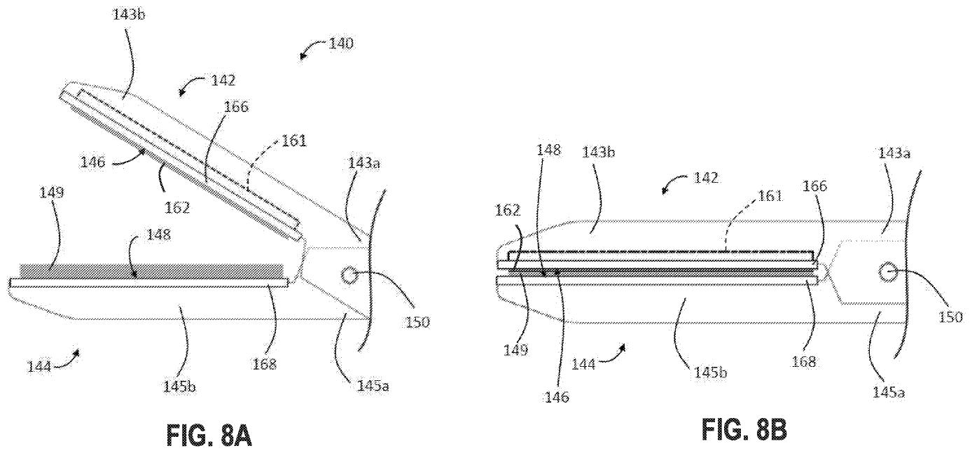

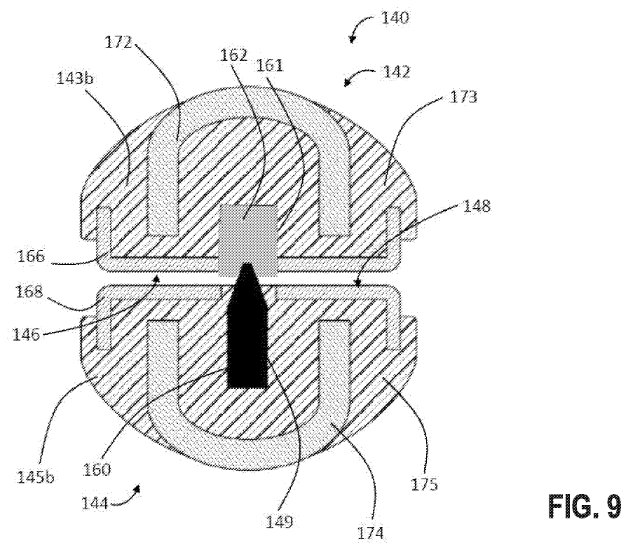

In configurations, jaw member 144 supports a longitudinally extending cutting electrode 149 in a slot 160 defined through tissue contacting surface 148 and a portion of distal body 145b of jaw member 144, while jaw member 142 includes a compression pad 162 (FIGS. 8A-9) disposed in a slot 161 (FIG. 9) defined through tissue contacting surface 146 and a portion of distal body 143b of jaw member 142. In such aspects, in the approximated position of jaw members 142, 144, cutting electrode 149 is urged into contact with compression pad 162 (FIGS. 8A-9) to grasp (and, in aspects, tension) tissue therebetween. Cutting electrode 149 may then be energized to cut the tissue disposed between cutting electrode 149 and compression pad 162 (FIGS. 8A-9). Cutting electrode 149 may additionally or alternatively be used to cut tissue in an open jaw configuration, e.g., with jaw members 142, 144 disposed in the spaced apart position. Cutting electrode 149 may be configured to be energized with monopolar Radio Frequency (RF) energy from a surgical generator (not shown) to conduct RF energy to tissue to cut the tissue, wherein the RF energy is returned to the generator to complete the circuit via a remote return device such as a return pad (not shown) or a local return device such as another portion of end effector assembly 140 or a separate instrument (not shown), e.g., a tenaculum, a probe, etc. Alternatively or additionally, cutting electrode 149 may be energized with bipolar RF energy wherein energy conducted from cutting electrode 149 to tissue is returned via either or both of tissue contacting surfaces 146, 148 of jaw members 142, 144, respectively, or other suitable local return device.

Referring still to FIG. 5, a drive rod 1484 is operably coupled to cam slot assembly 152 of end effector assembly 140, e.g., engaged with the cam pin thereof, such that longitudinal actuation of drive rod 1484 pivots jaw member 142 relative to jaw member 144 between the spaced apart and approximated positions. More specifically, urging drive rod 1484 proximally pivots jaw member 142 relative to jaw member 144 towards the approximated position while urging drive rod 1484 distally pivots jaw member 142 relative to jaw member 144 towards the spaced apart position. However, other suitable mechanisms and/or configurations for pivoting jaw member 142 relative to jaw member 144 between the spaced apart and approximated positions in response to selective actuation of drive rod 1484 are also contemplated. Drive rod 1484 extends proximally from end effector assembly 140 through shaft 130 and into housing 120 wherein drive rod 1484 is operably coupled with a jaw drive sub-assembly 1400 of actuation assembly 1100 (FIGS. 6A-6B) to enable selective actuation of end effector assembly 140 to grasp tissue therebetween and apply a jaw force within an appropriate jaw force range.

Tissue contacting surfaces 146, 148 of jaw members 142, 144, respectively, are at least partially formed from an electrically conductive material and are energizable to different potentials to enable the conduction of RF electrical energy through tissue grasped therebetween, although tissue contacting surfaces 146, 148 may alternatively be configured to supply any suitable energy, e.g., thermal, microwave, light, ultrasonic, etc., through tissue grasped therebetween for energy based tissue treatment. Instrument 110 defines a conductive pathway (not shown) through housing 120 and shaft 130 to end effector assembly 140 that may include lead wires, contacts, and/or electrically conductive components to enable electrical connection of tissue contacting surfaces 146, 148 of jaw members 142, 144, respectively, and cutting electrode 149 (FIG. 9) to an energy source (not shown), e.g., an electrosurgical generator, for supplying energy to tissue contacting surfaces 146, 148 to treat, e.g., seal, tissue grasped between tissue contacting surfaces 146, 148 and to supply energy to cutting electrode 149 (FIG. 9) to treat, e.g., cut, tissue grasped between tissue contacting surfaces 146, 148 or otherwise positioned adjacent to cutting electrode 149 (FIG. 9).

With additional reference to FIGS. 6A-7, as noted above, actuation assembly 1100 is disposed within housing 120 and includes an articulation sub-assembly 1200, and a jaw drive sub-assembly 1400. Articulation sub-assembly 1200 is operably coupled between first and second input couplers 1110, 1120, respectively, of actuation assembly 1100 and articulation cables 138 (FIG. 5) such that, upon receipt of appropriate inputs into first and/or second input couplers 1110, 1120, articulation sub-assembly 1200 manipulates cables 138 (FIG. 5) to articulate end effector assembly 140 in a desired direction, e.g., to pitch and/or yaw end effector assembly 140. Articulation sub-assembly 1200 is described in greater detail below.

Jaw drive sub-assembly 1400 is operably coupled between fourth input coupler 1140 of actuation assembly 1100 and drive rod 1484 such that, upon receipt of appropriate input into fourth input coupler 1140, jaw drive sub-assembly 1400 pivots jaw members 142, 144 between the spaced apart and approximated positions to grasp tissue therebetween and apply a jaw force within an appropriate jaw force range.

Actuation assembly 1100 is configured to operably interface with a surgical robotic system, e.g., system 10 (FIG. 1), when instrument 110 is mounted on a robotic arm thereof, to enable robotic operation of actuation assembly 1100 to provide the above detailed functionality. That is, surgical robotic system 10 (FIG. 1) selectively provides inputs, e.g., rotational inputs to input couplers 1110-1140 of actuation assembly 1100 to articulate end effector assembly 140, grasp tissue between jaw members 142, 144, and/or cut tissue grasped between jaw members 142, 144. However, as noted above, it is also contemplated that actuation assembly 1100 be configured to interface with any other suitable surgical systems, e.g., a manual surgical handle, a powered surgical handle, etc.

Turning to FIGS. 8A-9, a distal portion of end effector assembly 140 is shown with jaw members 142, 144 disposed in the spaced apart position (FIG. 8A) and the approximated position (FIGS. 8B and 9). In aspects, either or both tissue contacting surfaces 146, 148 of jaw members 142, 144, respectively, are defined by respective tissue contacting plates 166, 168 disposed on the opposing surfaces of distal bodies 143b, 145b of jaw members 142, 144, respectively. As detailed above, jaw member 144 supports cutting electrode 149 in slot 160 defined through tissue contacting surface 148 (and through tissue contacting plate 168 and a portion of distal body 145b of jaw member 144), while jaw member 142 includes compression pad 162 disposed in slot 161 defined through tissue contacting surface 146 (and through tissue contacting plate 166 and a portion of distal body 143b of jaw member 142) and configured to oppose cutting electrode 149 in the approximated position of jaw members 142, 144 (FIGS. 8B and 9). Tissue contacting surfaces 146, 148 may define substantially U-shaped configurations wherein the slots 161, 160 defined therethrough terminate at positions proximally spaced from the distal ends of tissue contact surfaces 146, 148.

Cutting electrode 149 protrudes from jaw member 144 beyond tissue contacting plate 168 and towards jaw member 142. Compression pad 162 may protrude from tissue contacting surface 146 of tissue contacting plate 166 towards jaw member 144, may be recessed relative to tissue contacting surface 146 of tissue contacting plate 166, or may be substantially flush with tissue contacting surface 146 of tissue contacting plate 166. Compression pad 162 and jaw member 142 are configured, in conjunction with cutting electrode 149, such that, in the approximated position of jaw members 142, 144 (see FIGS. 8B and 9), cutting electrode 149 is urged into and at least partially compresses compression pad 162 (with tissue grasped therebetween), thus facilitating electrical tissue cutting upon activation of cutting electrode 149. The contact between cutting electrode 149 and compression pad 162 may also maintain a spacing between tissue contacting surfaces 146, 148 to inhibit electrical shorting via contact therebetween.

Either or both jaw members 142, 144 may include a structural jaw support 172, 174 defining the respective proximal flange 143a, 145a of the jaw member 142, 144 and extending into the respective distal body 143b, 145b. In such configurations, distal body 143b, 145b of either or both jaw members 142, 144 may further include jaw housings 173, 175 surrounding structural jaw supports 172, 174 and supporting tissue contacting plates 166, 168, respectively, thereon. Jaw housings 173, 175 may be formed from insulative materials and, in aspects, may be overmolded about jaw supports 172, 174 and a portion of tissue contacting plates 166, 168 to form jaw members 142, 144 and secure the components thereof to one another. In other configurations, jaw housings 173, 175 are conductive and electrically isolated from the other components of jaw members 142, 144 via suitable insulation. Alternatively or additionally, either or both jaw members 142, 144 may be formed from a monolithic, electrically conductive piece of material defining the structural jaw support, tissue contacting surface, and jaw housing thereof. At least a portion of the jaw housing, in such configurations, may be coated with an insulative material. Further, with respect to configurations where jaw member 144 is formed from a monolithic piece of material, cutting electrode 149 may be electrically isolated from the remainder of jaw member 144, e.g., via an insulator disposed therebetween. Thus, as utilized herein, reference to jaw housings 173, 175 includes insulative jaw housings, conductive jaw housings, and/or monolithic jaw structures defining jaw housings. Further, both jaw members 142, 144 may be similarly configured or may define different configurations, such as any combination of the jaw configurations detailed herein.

Continuing with reference to FIGS. 8A-9, as noted above, compression pad 162 and jaw member 142 are configured to facilitate electrical cutting of tissue grasped between jaw members 142, 144 upon activation of cutting electrode 149. More specifically, compression pad 162 is at least partially resiliently compressible and defines a suitable durometer, suitable durometer profile (e.g., with portions having different durometers), and/or suitable size and shape configuration to facilitate grasping tissue between compression pad 162 and cutting electrode 149 with sufficient force (and, in aspects, suitable tension) to enable effective and efficient electrical cutting of tissue upon activation of cutting electrode 149. Various configurations of compression pad 162 to facilitate effective and efficient electrical cutting of tissue upon activation of cutting electrode 149 are detailed below.

Compression pad 162 may define a substantially uniform shape and/or material(s) along the length of compression pad 162, across the width of compression pad 162, and/or through the depth of compression pad 162 such that compression pad 162 exhibits substantially similar properties, e.g., durometer, across these dimension(s). Alternatively, compression pad 162 may define a varied shape and/or be formed from different materials along the length of compression pad 162, across the width of compression pad 162, and/or through the depth of compression pad 162 such that compression pad 162 defines a particular durometer profile across these dimension(s).

Compression pad 162 may be formed from any suitable resiliently compressible material such as, for example, silicone or polytetrafluoroethylene (PTFE). Other suitable resiliently compressible materials having sufficient thermal properties are also contemplated for forming at least a portion of compression pad 162 such as, for example, resiliently compressible materials capable of withstanding temperatures of, in aspects, at least 200° C.; in other aspects, of at least 240° C.; or, in still other aspects, of at least 260° C. In aspects, compression pad 162 is formed from an overmold or injection moldable material or materials.

Compression pad 162, in aspects, may be formed from a single material or a substantially homogeneous mixture of materials. Alternatively or additionally, compression pad 162 may include filler materials disposed thereon (e.g., on the tissue contacting surface thereof) or therein (e.g., uniformly or non-uniformly distributed throughout compression pad 162). Such filler materials include, without limitation: calcium carbonate, talc, silica, wollastonite, clay, calcium sulfate fibers, mica, glass beads, and alumina trihydrate. Filler materials such as those noted above provide texture and/or roughness which increases gripping and reduces slippage of tissue grasped between compression pad 162 and cutting electrode 149 (FIG. 9). Such filler materials may also increase the effective durometer of compression pad 162, at least in the portions of compression pad 162 where such filler materials are provided. Other suitable filler materials or structures incorporated into compression pad 162 may provide increased structural support uniformly or selectively across one or more dimensions of compression pad 162 to achieve a particular effective durometer or effective durometer profile of compression pad 162 and/or for other purposes (e.g., texturing). Likewise, the selective removal of material from on or within compression pad 162 and/or the formation of compression pad 162 with voids, channels, cut-outs, etc. uniformly or selectively across one or more dimensions of compression pad 162 may also be utilized to achieve a particular effective durometer or effective durometer profile of compression pad 162 and/or for other purposes (e.g., texturing).

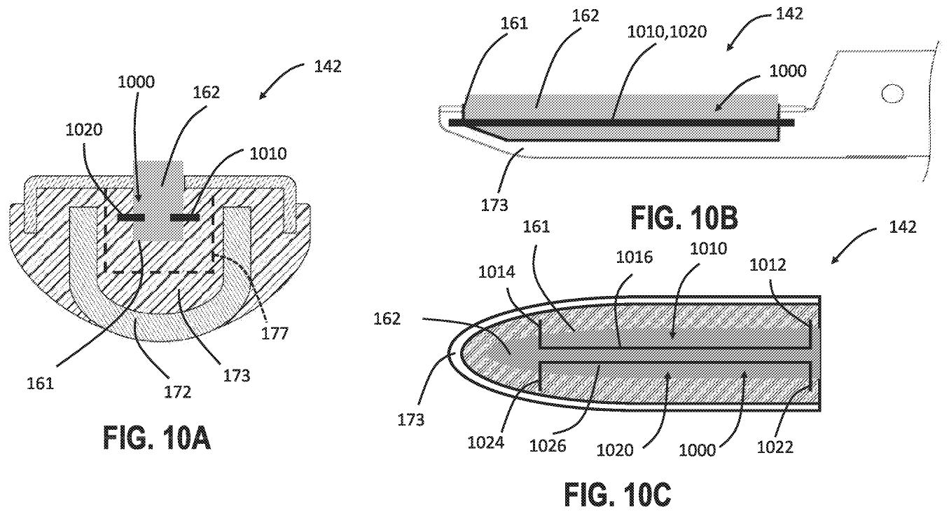

In order to provide the desired effective durometer or effective durometer profile, and/or to contribute thereto, compression pad 162 is appropriately engaged within jaw member 142. Various aspects and features facilitating the engagement of compression pad 162 within jaw member 142 to provide the desired effective durometer or effective durometer profile and, thus, to enable effective and efficient electrical cutting of tissue upon activation of cutting electrode 149 are detailed below with reference to FIGS. 10A-18. To the extent consistent, any or all of these aspects and features may be used in any suitable combination with any or all of the other aspects and features.

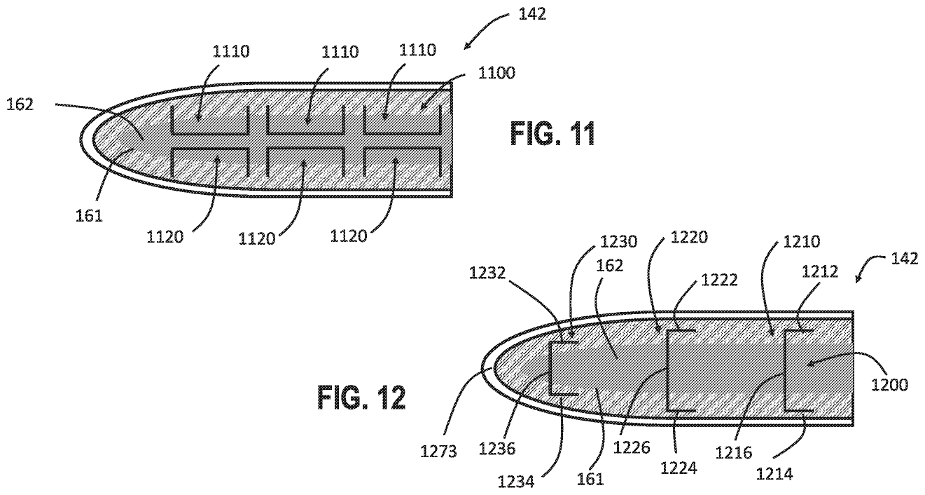

Referring to FIGS. 10A-10C, jaw member 142 is shown wherein an engagement configuration 1000 is provided including one or more retainers 1010, 1020 configured to facilitate the engagement of compression pad 162 within jaw member 142. More specifically, first and second retainers 1010, 1020 are fixed relative to jaw housing 173 and extend into slot 161. First and second retainers 1010, 1020 may be formed monolithically with jaw housing 173 or a portion thereof (e.g., an insulative portion of jaw housing 173, structural jaw support 172, or an insulative insert disposed within jaw housing 173 or structural jaw support 172) or may be engaged within jaw housing 173 in any suitable manner such as, for example, via mechanical engagement, press fitting, adhesion, molding, welding, and/or in any other suitable manner. Further, first and second retainers 1010, 1020 may be formed from a metal (including metal alloys), a polymeric material, or any other suitable material and may define a cylindrical cross-sectional configuration (e.g., a rod or wire configuration), a flat or plate-like cross-sectional configuration, a rectangular cross-sectional configuration, or any other suitable configuration including varied cross-sectional configurations along the length thereof. First and second retainers 1010, 1020 may be formed by molding, stamping, extrusion, or in any other suitable manner. In addition, although two retainers 1010, 1020 are shown and described with reference to FIGS. 10A-10C, it is contemplated that other suitable number and/or configurations of retainers may be provided such as, for example, detailed below with reference to FIGS. 11-14. Further, first and second retainers 1010, 1020 may be connected to one another (e.g., formed as a single component) or may be separate components (as shown).

Each of first and second retainers 1010, 1020 includes a proximal leg 1012, 1022, a distal leg 1014, 1024, and a body 1016, 1026 extending between the respective proximal and distal legs 1012, 1022 and 1014, 1024. Proximal and distal legs 1012, 1014 of first retainer 1010 are fixed within jaw housing 173 and extend transversely into slot 161 in a first direction while proximal and distal legs 1022, 1024 of second retainer 1020 are fixed within jaw housing 173 and extend substantially transversely into slot 161 in an opposing, second, opposite direction. Bodies 1016, 1026 extend substantially longitudinally within slot 161 in substantially parallel, spaced apart relation relative to one another and the side walls that define slot 161. In this manner, slot 161 defines free space surrounding the entireties of bodies 1016, 1026 and at least portions of legs 1012, 1014, 1022, 1024. In configurations where first and second retainers 1010, 1020 define plate-like configurations, the area defined between the legs 1012, 1014, 1022, 1024 and bodies 1016, 1026 may be fully occupied by the material of the retainer 1010, 1020, e.g., such that free space is not defined between the bodies 1016, 1026 and side walls that define slot 161. Further, it is contemplated that legs 1012, 1022 and 1014, 1024 of retainers 1010, 1020, respectively, extend in other suitable directions such as, for example, vertically upwardly or downwardly, at angles, etc., and may extend in the same or different directions from the other legs 1012, 1022 and 1014, 1024.

The above detailed arrangement of first and second retainers 1010, 1020 enables molding of compression pad 162 into slot 161 such that the mold material forming compression pad 162 occupies the free space defined by slot 161 thus surrounding bodies 1016, 1026 and at least portions of legs 1012, 1014, 1022, 1024. As the mold material is cured to form compression pad 162, compression pad 162 captures first and second retainers 1010, 1020 within compression pad 162 to thereby securely engage compression pad 162 relative to first and second retainers 1010, 1020 and, thus, securely engage and retain compression pad 162 within slot 161.

As an alternative to molding compression pad 162 into slot 161 and about first and second retainers 1010, 1020, compression pad 162 may be pre-formed with suitable features (e.g., slots, slits, perforations, etc.) and inserted into slot 161 to enable the features of compression pad 162 to receive or otherwise engage first and second retainers 1010, 1020 to thereby securely engage compression pad 162 relative to first and second retainers 1010, 1020 and, thus, securely engage and retain compression pad 162 within slot 161.

Further still, in aspects, rather than molding and/or inserting compression pad 162 directly into jaw member 142, slot 161 may be formed within an insulative insert 177 (FIG. 10A) configured for positioning within jaw housing 173 and/or jaw support 172 (see FIG. 9) and compression pad 162 may be secured therein with the use of first and second retainers 1010, 1020 according to any of the aspects detailed herein. Insulative insert 177 may then be secured within jaw member 142 (e.g., jaw housing 173 and/or jaw support 173 (see FIG. 9)) via adhesion, mechanical engagement, overmolding or in any other suitable manner. In aspects, first and second retainers 1010, 1020 may be formed integrally as part of insert 177.

Continuing with reference to FIGS. 10A-10C, first and second retainers 1010, 1020 of engagement configuration 1000, in addition to providing for secure engagement of compression pad 162 within slot 161, may also alter the effective durometer or effective durometer profile of compression pad 162. For example, the effective durometer of compression pad 162 may be relatively greater towards the longitudinal sides of compression pad 162 (where first and second retainers 1010, 1020 extend through compression pad 162) and relatively less in the longitudinal center of compression pad 162 (which is devoid of first and second retainers 1010, 1020). Different configuration of retainers such as, for example, detailed below with reference to FIGS. 11-14, may be provided to achieve any suitable effective durometer or effective durometer profile of compression pad 162.

With reference to FIG. 11, another engagement configuration 1100 to facilitate the engagement of compression pad 162 within jaw member 142 is shown. Engagement configuration 1100 is similar to and may include any of the features of engagement configuration 1000 (FIGS. 10A-10C) except that, rather than first and second retainers as with engagement configuration 1000 (FIGS. 10A-10C), engagement configuration 1100 includes a plurality of first retainers 1110 spaced longitudinally along slot 161 and extending transversely into slot 161 in a first direction and a plurality of second retainers 1120 spaced longitudinally along slot 161 and extending transversely into slot 161 in an opposing, second, opposite direction.

FIG. 12 illustrates another engagement configuration 1200 to facilitate the engagement of compression pad 162 within jaw member 142. Engagement configuration 1200 is similar to and may include any of the features of engagement configuration 1000 (FIGS. 10A-10C) except that, rather than the bodies of the retainers extending longitudinally within slot 161 as with engagement configuration 1000 (FIGS. 10A-10C), engagement configuration 1200 includes a plurality of retainers 1210, 1220, 1230 oriented such that bodies 1216, 1226, 1236 of respective retainers 1210, 1220, 1230 extend transversely across slot 161 in longitudinally spaced relation relative to one another along the length of slot 161. In this configuration, the legs 1212 and 1214, 1222 and 1224, 1232 and 1234 of each retainer 1210, 1220, 1230, respectively, may be fixed within jaw housing 173 in longitudinally oriented position or may define any other suitable configuration.

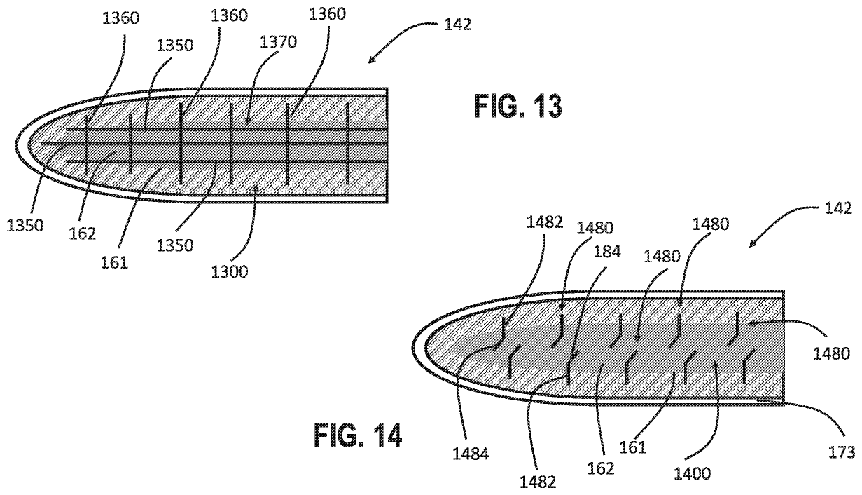

Turning to FIG. 13, still another engagement configuration 1300 to facilitate the engagement of compression pad 162 within jaw member 142 is shown. Engagement configuration 1300 is similar to a combination of engagement configurations 1100, 1200 (FIGS. 11 and 12, respectively) and may include any of the features of any of the engagement configurations detailed herein. Engagement configuration 1300, more specifically, includes one or more longitudinally extending retainers 1350 and one or more transversely extending retainers 1360 (and/or retainers extending in other suitable directions) to define a matrix of retainers 1370. Longitudinally extending retainers 1350 and transversely extending retainers 1360 may be joined to one another at the intersections thereof, may contact one another at the intersections thereof, or may be offset (e.g., vertically) to intersect one another without contact therebetween.

Referring to FIG. 14, yet another engagement configuration 1400 to facilitate the engagement of compression pad 162 within jaw member 142 is shown. Engagement configuration 1400 may be similar to and may include any of the features of any of the engagement configurations detailed herein except that, rather than each retainer extending completely through or across slot 161 (see, e.g., FIGS. 10A-13), engagement configuration 1400 includes a plurality of retainers 1480 each include a first end 1482 fixed within jaw housing 173 and extending into slot 161 to a second, free end 1484. The portions of retainers 1480 extending into slot 161 may include one or more angled portions (as shown), curved portions, and/or other suitable features to facilitate secure engagement and retention of compression pad 162 within slot 161.

FIGS. 11-14 illustrate engagement configurations 1100-1400 that, similarly as detailed above with respect to configuration 1000, may also alter the effective durometer or effective durometer profile of compression pad 162 in addition to providing for secure engagement of compression pad 162 within slot 161. Thus, any suitable effective durometer or effective durometer profile of compression pad 162 may be achieved.

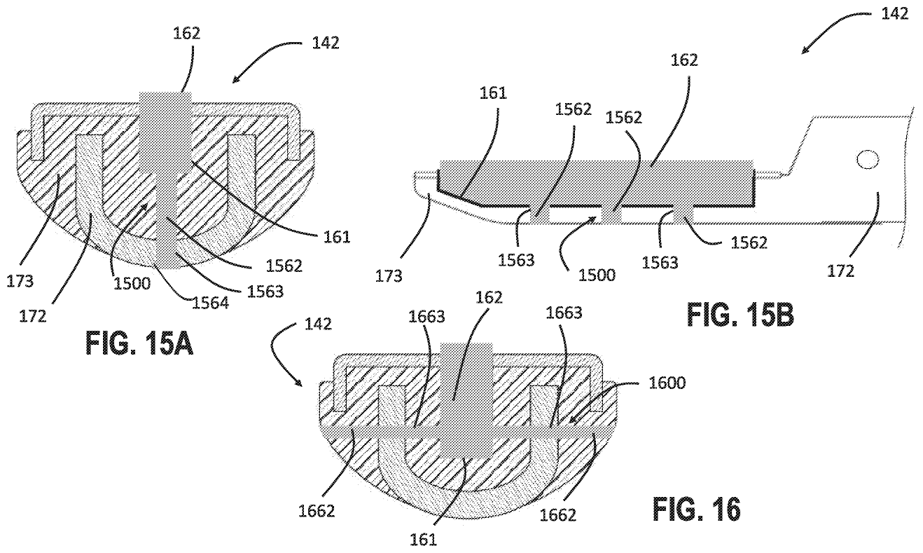

With reference to FIGS. 15A and 15B, engagement configuration 1500 facilitates the engagement of compression pad 162 within jaw member 142. Engagement configuration 1500 includes one or more channels 1563 defined through at least a portion of jaw member 142 and communicating with slot 161. More specifically, the one or more channels 1563 may include a plurality of substantially vertically oriented channels 1563 extending from the closed bottom of slot 161 at least partially through jaw member 142. As shown in FIGS. 15A and 15B, for example, the plurality of substantially vertically oriented channels 1563 may be longitudinally spaced along the length of slot 161. The one or more channels 1563 may extend partially or fully through the entirety of jaw member 142, through multiple different parts of jaw member 142, or just through a single part of jaw member 142. For example, the one or more channels 1563 may extend through an insulative body portion of jaw housing 173, jaw support 172, and/or an outer insulative cover of jaw housing 173. In aspects, the one or more channels 1563 includes a countersink feature 1564 (e.g., an enlarged end portion) to facilitate retention of compression pad 162 once the mold material forming compression pad 162 is cured. Countersink features may additionally or alternatively be incorporated into other aspects detailed herein for similar purposes, e.g., to facilitate retention of compression pad 162.

The one or more channels 1563 may extend linearly or may define angled portions, curved portions, etc. The one or more channels 1563 may define uniform dimensions or may define varied dimensions such as, for example, to define wider or narrower portions. Further, although one row of channels 1563 is shown substantially centered transversely across jaw member 142, additional rows and/or positions of rows of channels 1563 are contemplated.

In use, since the one or more channels 1563 are disposed in communication with slot 161, as the mold material forming compression pad 162 flows into slot 161, some of the mold material continued through slot 161 and into the one or more channels 1563. Thus, once the mold material is cured to form compression pad 162, compression pad 162 not only occupies slot 161 but also includes extensions 1562 that extend partially or fully through the one or more channels 1563 (and, in aspects, countersink features thereof), thus increasing the engagement and retention of compression pad 162 within slot 161.

In aspects, the mold material forming compression pad 162 may flow from slot 161 through the one or more channels 1563 to define other features of jaw member 142. For example, compression pad 162 and an outer insulative jaw cover may be formed in a single-shot mold process whereby compression pad 162 and the outer insulative jaw cover are connected via the mold material extending from slot 161 through the one or more channels 1563 to the outer surface of jaw member 142 to define the insulative jaw cover.

As shown in FIG. 16, in addition or as an alternative to substantially vertically extending channels (see FIG. 15), an engagement configuration 1600 may be provided with one or more horizontally extending channels 1663 defined at least partially through jaw member 142 and disposed in communication with slot 161. The mold material forming compression pad 162 may flow through slot 161 and channels 1663 such that, once cured, compression pad 162 is formed to include extensions 1662 extending partially or fully through the one or more channels 1663. In alternative or additional aspects, a structural element, e.g., a metal pin or pre-formed plastic pin, may be disposed within channels 1663 to form at least a portion of and/or providing structural support to extensions 1662. Engagement configuration 1600 may otherwise be similar to and include any of the features of engagement configuration 1500 (FIG. 15) as detailed above.

Referring to FIGS. 15A-16, in addition to providing for secure engagement of compression pad 162 within slot 161, engagement configurations 1500, 1600 may also alter the effective durometer or effective durometer profile of compression pad 162. Thus, engagement configurations 1500, 1600 may be provided to achieve any suitable effective durometer or effective durometer profile of compression pad 162.

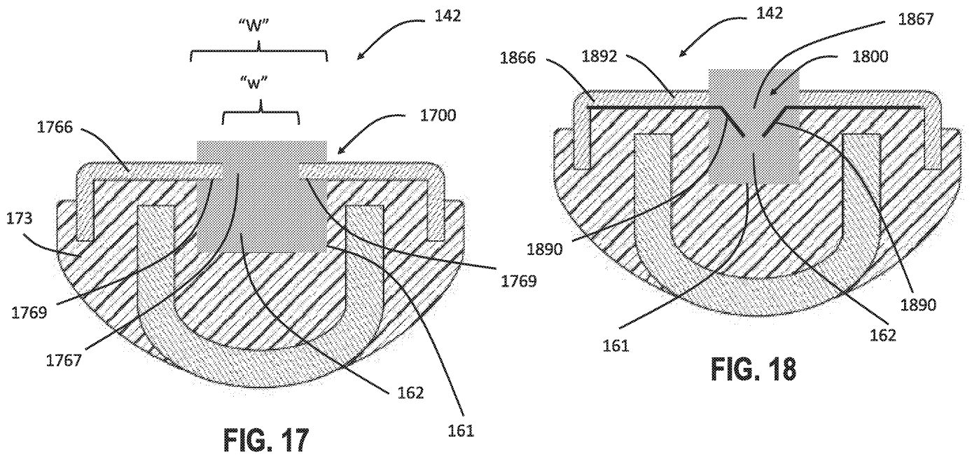

Turning to FIG. 17, another engagement configuration 1700 provided in accordance with the present disclosure to facilitate the engagement of compression pad 162 within jaw member 142 is shown. Slot 161 of jaw member 142 has a width “W” defined by jaw housing 173 or an insert disposed within jaw housing 173. Further, tissue contacting plate 1766 defines an opening 1767 in communication with slot 161. Opening 1767 defines a width “w” less than the width “W” such that tissue contacting plate 1666 defines overhangs 1769 that overhang slot 161 on either side thereof at the open top of slot 161, e.g., such that an underside of tissue contacting plate 1766 is exposed over slot 161. In this manner, overhangs 1769 of tissue contacting plate 1766 define retainers to facilitate retention and engagement of compression pad 162 within slot 161, e.g., similarly as detailed above with reference to FIGS. 10A-10C.

Overhangs 1769 of tissue contacting plate 1766, more specifically, enable molding of compression pad 162 into slot 161 such that the mold material forming compression pad 162 fills slot 161, surrounds overhangs 1769, and extends beyond tissue contacting plate 1766 towards jaw member 144 (see FIG. 9). As the mold material is cured to form compression pad 162, compression pad 162 captures overhangs 1769 within compression pad 162 to thereby securely engage compression pad 162 relative to tissue contacting plate 1766 and, thus, securely engage and retain compression pad 162 within slot 161. In aspects, compression pad 162 need not extend outwardly over the tissue contacting surface of tissue contacting plate 1766 but may define an upper portion having a width substantially equal to width “w” such that the upper portion of compression pad 162 extends vertically to, at least partially through, or vertically from opening 1767 of tissue contacting plate 1766.

As an alternative to molding compression pad 162 into slot 161 and about overhangs 1769, compression pad 162 may be pre-formed with suitable features (e.g., slots, slits, perforations, etc.) and inserted into slot 161 to enable the features of compression pad 162 to receive or otherwise engage overhangs 1769 to thereby securely engage compression pad 162 relative to overhangs 1769 and, thus, securely engage and retain compression pad 162 within slot 161.

Overhangs 1769, in addition to providing for secure engagement of compression pad 162 within slot 161, may also alter the effective durometer or effective durometer profile of compression pad 162. For example, the effective durometer of compression pad 162 may be relatively greater towards the longitudinal sides of compression pad 162 (where overhangs 1769 extend into compression pad 162) and relatively less in the longitudinal center of compression pad 162 (which is devoid of overhangs 1769). Any other suitable effective durometer or effective durometer profile of compression pad 162 may also be provided.

Referring to FIG. 18, another engagement configuration 1800 provided in accordance with the present disclosure to facilitate the engagement of compression pad 162 within jaw member 142 is shown. Engagement configuration 1800 is similar to engagement configuration 1700 (FIG. 17) and may include any of the features thereof, except as contradicted below. With respect to engagement configuration 1800, rather than the body of tissue contacting plate extending continuously inwardly to overhang slot 161 as detailed above with respect to engagement configuration 1700 (FG. 17), engagement configuration 1800 includes first and second tangs 1890 extending inwardly from body 1892 of tissue contacting plate 1866 on either side of opening 1867 of tissue contacting plate 1866. Tangs 1890 differ from body 1892 of tissue contacting plate 1866 in one or more of thickness, orientation, shape, texture, etc. Tangs 1890 may be formed monolithically with body 1892 of tissue contacting plate 1866 in a single manufacturing process, e.g., stamping, or may be subsequently formed from body 1892 of tissue contacting plate 1866, e.g., via bending the inner ends of body 1892 of tissue contacting plate 1866 to form tangs 1890. It is also contemplated that tangs 1890 be separately formed and subsequently attached to body 1892 of tissue contacting plate 1866. In other aspects, tissue contacting plate 1866 is formed form a plurality of layers and wherein at least one but not all layers extend inwardly from either inner side of tissue contacting plate 1866 to define tangs 1890. Other configurations are also contemplated.

Similarly as detailed above with respect to overhangs 1769 (FIG. 17), tangs 1890, in addition to providing for secure engagement of compression pad 162 within slot 161, may also alter the effective durometer or effective durometer profile of compression pad 162. For example, the effective durometer of compression pad 162 may be relatively greater towards the longitudinal sides of compression pad 162 (where tangs 1890 extend into compression pad 162) and relatively less in the longitudinal center of compression pad 162 (which is devoid of tangs 1890). Any other suitable effective durometer or effective durometer profile of compression pad 162 may also be provided.

Aspects of this disclosure may be further described by reference to the following numbered paragraphs:

1. A surgical end effector assembly, comprising: first and second jaw members including respective first and second tissue contacting surfaces, at least one of the first or second jaw members movable relative to the other of the first or second jaw members between a spaced apart position and an approximated position, wherein the second jaw member includes a cutting electrode extending from the second jaw member towards the first jaw member, and wherein the first jaw member includes a jaw body defining a slot and at least one retainer fixed relative to the jaw body and extending into the slot, the first jaw member further including a compression pad at least partially disposed within the slot and about the at least one retainer to capture at least a portion of the at least one retainer within the compression pad and engage the compression pad at least partially within the jaw body, and wherein, in the approximated position, the cutting electrode and the compression pad are configured to grasp tissue therebetween.

2. The surgical end effector assembly according to paragraph 1, wherein the at least one retainer includes a first retainer extending inwardly into the slot from a first side of the slot and a second retainer extending inwardly into the slot from a second, opposite side of the slot.

3. The surgical end effector assembly according to paragraph 2, wherein the at least one retainer includes a plurality of first retainers and a plurality of second retainers.

4. The surgical end effector assembly according to paragraph 2, wherein each of the first and second retainers includes a body extending substantially longitudinally through the slot, wherein the bodies of the first and second retainers are captured within the compression pad.

5. The surgical end effector assembly according to any preceding paragraph, wherein the at least one retainer includes a body extending substantially transversely across the slot, and wherein the body of the at least one retainer is captured within the compression pad.

6. The surgical end effector assembly according to any preceding paragraph, wherein the at least one retainer varies an effective durometer of the compression pad in at least one dimension thereof.

7. The surgical end effector assembly according to any preceding paragraph, wherein the jaw body of the first jaw member includes a jaw housing and a jaw insert disposed within the jaw housing, wherein the jaw insert defines the slot and wherein the at least one retainer is fixed relative to the jaw insert.

8. The surgical end effector assembly according to any preceding paragraph, wherein the at least one retainer includes at least one wire.

9. A surgical end effector assembly, comprising: first and second jaw members including respective first and second tissue contacting surfaces, at least one of the first or second jaw members movable relative to the other of the first or second jaw members between a spaced apart position and an approximated position, wherein the second jaw member includes a cutting electrode extending from the second jaw member towards the first jaw member, wherein the first jaw member includes a jaw body defining a slot, a tissue contacting plate defining the first tissue contacting surface and extending along first and second longitudinal sides of the slot, and a compression pad, wherein first and second overhangs extend from the tissue contacting plate into the slot from the respective first and second longitudinal sides of the slot, and wherein the compression pad is at least partially disposed within the slot, and wherein, in the approximated position, the cutting electrode and the compression pad are configured to grasp tissue therebetween.

10. The surgical end effector assembly according to paragraph 9, wherein the compression pad captures the first and second overhangs therein and extends over a portion of the first tissue contacting surface.

11. The surgical end effector assembly according to paragraph 9 or 10, wherein the first and second overhangs extend uninterrupted from the tissue contacting plate in coplanar relation relative to the first tissue contacting surface.

12. The surgical end effector assembly according to any one of paragraphs 9-11, wherein the first and second overhangs are tangs, the tangs angled relative to the first tissue contacting surface.

13. The surgical end effector assembly according to any one of paragraphs 9-12, wherein the first and second overhangs are tangs defining a reduced thickness compared to the tissue contacting plate.

14. The surgical end effector assembly according to any one of paragraphs 9-13, wherein the first and second overhangs vary an effective durometer of the compression pad in at least one dimension thereof.

15. A surgical end effector assembly, comprising: first and second jaw members including respective first and second tissue contacting surfaces, at least one of the first or second jaw members movable relative to the other of the first or second jaw members between a spaced apart position and an approximated position, wherein the second jaw member includes a cutting electrode extending from the second jaw member towards the first jaw member, wherein the first jaw member includes a jaw body defining a slot and at least one channel in communication with the slot, the first jaw member further including a compression pad at least partially disposed within the slot and the at least one channel to engage the compression pad at least partially within the jaw body, and wherein, in the approximated position, the cutting electrode and the compression pad are configured to grasp tissue therebetween.

16. The surgical end effector assembly according to paragraph 15, wherein the at least one channel includes at least one channel extending from a bottom of the slot.

17. The surgical end effector assembly according to paragraph 15 or 16, wherein the at least one channel includes a plurality of channels extending from a bottom of the slot in longitudinally spaced relation relative to one another.

18. The surgical end effector assembly according to any one of paragraphs 15-17, wherein the at least one channel includes at least one channel extending from a side of the slot.

19. The surgical end effector assembly according to any one of paragraphs 15-18, wherein the at least one channel includes a pair of channels extending from opposing sides of the slot.

20. The surgical end effector assembly according to any one of paragraphs 15-19, wherein the at least one channel varies an effective durometer of the compression pad in at least one dimension thereof.

While several aspects of this disclosure have been shown in the drawings, it is not intended that this disclosure be limited thereto, as it is intended that the disclosure be as broad in scope as the art will allow and that the specification be read likewise. Therefore, the above description should not be construed as limiting, but merely as exemplifications of particular aspects. Those skilled in the art will envision other modifications within the scope and spirit of the claims appended hereto.

1. A surgical end effector assembly (140), comprising:
first and second jaw members (142, 144) including respective first and second tissue contacting surfaces (146, 148), at least one of the first or second jaw members (142, 144) movable relative to the other of the first or second jaw members (142, 144) between a spaced apart position and an approximated position, wherein the second jaw member (144) includes a cutting electrode (149) extending from the second jaw member (144) towards the first jaw member (142), characterized in that:
the first jaw member (142) includes a jaw body (143b) defining a slot (161) and at least one retainer fixed relative to the jaw body (143b) and extending into the slot (161), the first jaw member (142) further including a compression pad (162) at least partially disposed within the slot (161) and about the at least one retainer (1010, 1020, 1110, 1120, 1210, 1220, 1230, 1350, 1360, 1370, 1480, 1769, 1890) to capture at least a portion of the at least one retainer (1010, 1020, 1110, 1120, 1210, 1220, 1230, 1350, 1360, 1370, 1480, 1769, 1890) within the compression pad (162) and engage the compression pad (162) at least partially within the jaw body (143b), wherein, in the approximated position, the cutting electrode (149) and the compression pad (162) are configured to grasp tissue therebetween.

2. The surgical end effector assembly (140) according to claim 1, wherein the at least one retainer includes a first retainer (1010, 1110, 1769, 1890) extending inwardly into the slot (161) from a first side of the slot (161) and a second retainer (1020, 1120, 1769, 1890) extending inwardly into the slot (161) from a second, opposite side of the slot (161).

3. The surgical end effector assembly (140) according to claim 2, wherein the at least one retainer includes a plurality of first retainers (1110) and a plurality of second retainers (1120).

4. The surgical end effector assembly (140) according to claim 2 or 3, wherein each of the first and second retainers (1010, 1020, 1110, 1120) includes a body extending substantially longitudinally through the slot (161), wherein the bodies of the first and second retainers (1010, 1020, 1110, 1120) are captured within the compression pad (162).

5. The surgical end effector assembly (140) according to claim 1, wherein the at least one retainer (1210, 1220, 1230) includes a body extending substantially transversely across the slot (161), and wherein the body of the at least one retainer (1210, 1220, 1230) is captured within the compression pad (162).

6. The surgical end effector assembly (140) according to any preceding claim, wherein the at least one retainer (1010, 1020, 1110, 1120, 1210, 1220, 1230, 1350, 1360, 1370, 1480, 1769, 1890) varies an effective durometer of the compression pad (162) in at least one dimension thereof.

7. The surgical end effector assembly (140) according to any preceding claim, wherein the jaw body (143b) of the first jaw member (142) includes a jaw housing (173) and a jaw insert (177) disposed within the jaw housing (173), wherein the jaw insert (177) defines the slot (161) and wherein the at least one retainer is fixed relative to the jaw insert (177).

8. The surgical end effector assembly (140) according to any preceding claim, wherein the at least one retainer (1010, 1020, 1110, 1120, 1210, 1220, 1230, 1350, 1360, 1370, 1480, 1890) includes at least one wire.

9. The surgical end effector assembly (140) according to claim 1, wherein the first jaw member (142) includes a tissue contacting plate (1766, 1866) defining the first tissue contacting surface (146) and extending along first and second longitudinal sides of the slot (161), and wherein first and second overhangs (1769, 1890) extend from the tissue contacting plate into the slot (161) to define the at least one retainer.

10. The surgical end effector assembly (140) according to claim 9, wherein the compression pad (162) captures the first and second overhangs (1769, 1890) therein and extends over a portion of the first tissue contacting surface (146).

11. The surgical end effector assembly (140) according to claim 9 or 10, wherein the first and second overhangs (1769) extend uninterrupted from the tissue contacting plate (1766) in coplanar relation relative to the first tissue contacting surface (146).

12. The surgical end effector assembly (140) according to claim 9 or 10, wherein the first and second overhangs are tangs (1890), the tangs (1890) angled relative to the first tissue contacting surface (146).

13. The surgical end effector assembly (140) according to claim 9, 10, or 12, wherein the first and second overhangs are tangs (1890) defining a reduced thickness compared to the tissue contacting plate (1866).

14. The surgical end effector assembly (140) according to any one of claims 9-13, wherein the first and second overhangs (1769, 1890) vary an effective durometer of the compression pad (162) in at least one dimension thereof.
